(19) United States
(12) Patent Application Publication
(10) Pub. No.: US 2021/0069782 A1
(43) Pub. Date: Mar. 11, 2021

(54) FINE PARTICLE PRODUCTION METHOD AND FINE PARTICLES

(71) Applicant: NISSHIN ENGINEERING INC., Chuo-ku, Tokyo (JP)

(72) Inventors: Shu WATANABE, Fujimino-city, Saitama (JP); Shiori SUEYASU, Fujimino-city, Saitama (JP); Keitaroh NAKAMURA, Fujimino-city, Saitama (JP)

(21) Appl. No.: 16/965,279

(22) PCT Filed: Jan. 10, 2019
(86) PCT No.: PCT/JP2019/000468

§ 371 Date: Jul. 27, 2020

(30) Foreign Application Priority Data
Jan. 26, 2018 (JP) 2018-011480

(51) Int. Cl. B22F 1/02; B22F 9/12

(52) U.S. Cl. CPC B22F 1/02; B22F 2301/30; B22F 2304/054; B22F 9/12

(57) ABSTRACT

Provided are: a fine particle production method that makes it possible to control the acidity, i.e., a surface property, of fine particles; and fine particles. A fine particle production method in which a raw material powder is used to produce fine particles by means of a gas phase method. The fine particle production method has a step for supplying an organic acid to raw material fine particles. The gas phase method is, for example, a thermal plasma method or a flame method. The fine particles have a surface coating that includes at least a carboxyl group.

### TECHNICAL FIELD

The present invention relates to a fine particle production method using a gas-phase process as well as fine particles, particularly to a fine particle production method and fine particles with the pH being controlled.

### BACKGROUND ART

At present, fine particles such as metal fine particles, oxide fine particles, nitride fine particles, carbide fine particles, oxynitride fine particles and resin fine particles are used in various applications. Fine particles are used in, for example, electrical insulation materials for insulating parts, functional materials for sensors, electrode materials for fuel cells, materials for cutting tools, materials for machining tools, sintered materials, conductive materials, and catalysts.

For instance, currently, a display device such as a liquid crystal display device is combined with a touch panel and used in tablet computers, smartphones and other devices, and the input operation using a touch panel has become widespread. Patent Literature 1 describes a method of producing silver fine particles usable in wiring of touch panels.

For instance, Patent Literature 2 describes a copper fine particle material that is sintered by heating at a temperature of not higher than 150° C. in a nitrogen atmosphere and has electric conductivity.

Further, Patent Literature 3 describes silicon/silicon carbide composite fine particles in which silicon fine particles are coated with silicon carbide, and Patent Literature 4 describes tungsten complex oxide particles.

### CITATION LIST

### Patent Literature

Patent Literature 1: WO 2016/080528

Patent Literature 2: JP 2016-14181 A

Patent Literature 3: JP 2011-213524 A

Patent Literature 4: WO 2015/186663

### SUMMARY OF INVENTION

### Technical Problems

As described above, fine particles are used in accordance with the intended use. However, even with the same composition, fine particles may be required to have a different property depending on the intended use. For instance, sometimes hydrophilicity is required, and sometimes hydrophobicity is required. In this case, control of the surface properties of fine particles, or the like, is necessary. As described above, various types of fine particles have been proposed, and in silicon/silicon carbide composite fine particles of Patent Literature 3 above, silicon fine particles are coated with silicon carbide, but the surface properties of the fine particles, such as hydrophilicity or hydrophobicity, are not controlled. There is a demand for fine particles having surface properties appropriate for the intended use under the current circumstances.

The present invention has been made to solve the problem that may arise from the conventional art and aims at providing a fine particle production method and fine particles that allow control of acidity which is one surface property of the fine particles.

### Solution to Problems

In order to attain the above object, the present invention provides a fine particle production method for producing fine particles using feedstock by means of a gas-phase process, the method comprising: a step of supplying an organic acid to raw material fine particles.

Preferably, the gas-phase process is a thermal plasma process or a flame process.

Preferably, in the step of supplying an organic acid, an aqueous solution containing the organic acid is sprayed to an atmosphere in which the organic acid is thermally decomposed.

Preferably, the organic acid consists only of C, O and H. Preferably, the organic acid is at least one of L-ascorbic acid, formic acid, glutaric acid, succinic acid, oxalic acid, DL-tartaric acid, lactose monohydrate, maltose monohydrate, maleic acid, D-mannite, citric acid, malic acid and malonic acid.

For example, the feedstock is powder of a metal other than silver, and metal fine particles are produced by means of the gas-phase process.

The present invention also provides fine particles each having a surface coating, wherein the surface coating contains at least a carboxyl group.

For example, the fine particles have a particle size of 1 to 100 nm.

The present invention also provides fine particles each having a surface coating, wherein the surface coating is constituted of an organic substance generated by thermal decomposition of an organic acid.

For example, the fine particles have a particle size of 1 to 100 nm.

Preferably, the organic acid consists only of C, O and H. Preferably, the organic acid is at least one of L-ascorbic acid, formic acid, glutaric acid, succinic acid, oxalic acid, DL-tartaric acid, lactose monohydrate, maltose monohydrate, maleic acid, D-mannite, citric acid, malic acid and malonic acid. Of these, the organic acid is preferably citric acid. Preferably, the fine particles are fine particles of a metal other than silver.

### Advantageous Effects of Invention

The present invention makes it possible to control surface properties, such as the pH, of fine particles.

The present invention also makes it possible to provide fine particles whose surface properties such as the pH are controlled.

### BRIEF DESCRIPTION OF DRAWINGS

FIG. 1 is a schematic view showing an example of a fine particle production apparatus that is used in a fine particle production method according to an embodiment of the invention.

FIG. 2 is a schematic view showing an example of fine particles according to an embodiment of the invention.

FIG. 3 is a graph showing analysis results of the crystal structures of metal fine particles obtained by the production method of the invention and metal fine particles obtained by a conventional production method, as analyzed by X-ray diffractometry.

### DESCRIPTION OF EMBODIMENTS

A fine particle production method and fine particles according to the present invention are described below in detail with reference to preferred embodiments shown in the accompanying drawings.

The fine particle production method of the invention is described below taking metal fine particles as an example of the fine particles.

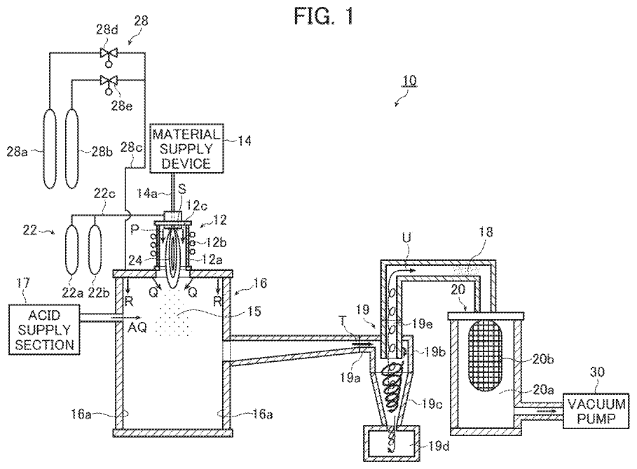

FIG. 1 is a schematic view showing an example of a fine particle production apparatus that is used in a fine particle production method according to an embodiment of the invention.

A fine particle production apparatus 10 (hereinafter referred to simply as “production apparatus 10”) shown in FIG. 1 is used to produce fine particles, e.g., metal fine particles. With the production apparatus 10, metal fine particles can be produced, and the pH of the metal fine particles can also be changed and controlled.

The fine particles are not particularly limited in type as long as they are fine particles, and the production apparatus 10 can produce fine particles other than the metal fine particles, namely, such fine particles as oxide fine particles, nitride fine particles, carbide fine particles, oxynitride fine particles and resin fine particles by changing the composition of the raw material.

The production apparatus 10 includes a plasma torch 12 generating thermal plasma, a material supply device 14 supplying feedstock of the fine particles into the plasma torch 12, a chamber 16 serving as a cooling tank for use in producing primary fine particles 15 of a feedstock-based material, an acid supply section 17, a cyclone 19 removing, from the produced primary fine particles 15 of the feedstock-based material, coarse particles having a particle size equal to or larger than an arbitrarily specified particle size, and a collecting section 20 collecting secondary fine particles 18 of the feedstock-based material having a desired particle size as obtained by classification by the cyclone 19. The primary fine particles 15 of the feedstock-based material before being supplied with an organic acid are particles in the middle of the production process of the fine particles of the invention, and the secondary fine particles 18 of the feedstock-based material are equivalent to the fine particles of the invention.

Various devices described in, for example, JP 2007-138287 A may be used for the material supply device 14, the chamber 16, the cyclone 19 and the collecting section 20. The primary fine particles 15 of a feedstock-based material are also simply called primary fine particles 15, and the secondary fine particles 18 of the feedstock-based material are also simply called secondary fine particles.

In this embodiment, metal powder is used as the feedstock in production of metal fine particles. The average particle size of the metal powder is appropriately set to allow easy evaporation of the powder in a thermal plasma flame and is, for example, not more than 100 μm, preferably not more than 10 μm, and more preferably not more than 5 μm.

The term “metal powder” includes single-composition metal powder and alloy powder containing plural compositions. The term “metal fine particles” includes single-composition metal fine particles and alloy fine particles made of an alloy containing plural compositions. For the metal powder, powders of metals except for silver, such as Cu, Si, Ni, W, Mo, Ti and Sn for instance, are preferably used. Metal fine particles of the above metals except for silver fine particles, for example, can be obtained by use of those metal powders.

In production of fine particles other than the metal fine particles, namely, such fine particles as oxide fine particles, nitride fine particles, carbide fine particles, oxynitride fine particles and resin fine particles as described above, powders such as oxide powder, nitride powder, carbide powder, oxynitride powder and resin powder are used as the feedstocks.

The plasma torch 12 is constituted of a quartz tube 12a and a coil 12b for high frequency oscillation surrounding the outside of the quartz tube. A supply tube 14a to be described later which is for supplying feedstock, e.g., metal powder for the metal fine particles, into the plasma torch 12 is provided on the top of the plasma torch 12 at the central part thereof. A plasma gas supply port 12c is formed in the peripheral portion of the supply tube 14a (on the same circumference). The plasma gas supply port 12c is in a ring shape.

A plasma gas supply source 22 is configured to supply plasma gas into the plasma torch 12 and for instance has a first gas supply section 22a and a second gas supply section 22b. The first gas supply section 22a and the second gas supply section 22b are connected to the plasma gas supply port 12c through piping 22c. Although not shown, the first gas supply section 22a and the second gas supply section 22b are each provided with a supply amount adjuster such as a valve for adjusting the supply amount. Plasma gas is supplied from the plasma gas supply source 22 into the plasma torch 12 through the plasma gas supply port 12c of ring shape in the direction indicated by arrow P and the direction indicated by arrow S.

For example, mixed gas of hydrogen gas and argon gas is used as plasma gas. In this case, hydrogen gas is stored in the first gas supply section 22a, while argon gas is stored in the second gas supply section 22b. Hydrogen gas is supplied from the first gas supply section 22a of the plasma gas supply source 22 and argon gas is supplied from the second gas supply section 22b thereof into the plasma torch 12 in the direction indicated by arrow P and the direction indicated by arrow S after passing through the plasma gas supply port 12c via the piping 22c. Argon gas may be solely supplied in the direction indicated by arrow P.

When a high frequency voltage is applied to the coil 12b for high frequency oscillation, a thermal plasma flame 24 is generated in the plasma torch 12.

It is necessary for the thermal plasma flame 24 to have a temperature higher than the boiling point of the metal powder (feedstock). The thermal plasma flame 24 preferably has a higher temperature because the metal powder (feedstock) is more easily converted into a gas phase state. However, there is no particular limitation on the temperature. For instance, the thermal plasma flame 24 may have a temperature of 6,000° C., and in theory, the temperature is deemed to reach around 10,000° C.

The ambient pressure inside the plasma torch 12 is preferably up to atmospheric pressure. The ambient pressure of up to atmospheric pressure is not particularly limited and is, for example, in the range of 0.5 to 100 kPa.

The periphery of the quartz tube 12a is surrounded by a concentrically formed tube (not shown), and cooling water is circulated between this tube and the quartz tube 12a to cool the quartz tube 12a with the water, thereby preventing the quartz tube 12a from having an excessively high temperature due to the thermal plasma flame 24 generated in the plasma torch 12.

The material supply device 14 is connected to the top of the plasma torch 12 through the supply tube 14a. The material supply device 14 is configured to supply the metal powder (feedstock) in a powdery form into the thermal plasma flame 24 in the plasma torch 12, for example.

For instance, as described above, the device disclosed in JP 2007-138287 A may be used as the material supply device 14 that supplies the metal powder (feedstock) in a powdery form. In this case, the material supply device 14 includes, for example, a storage tank (not shown) storing the metal powder (feedstock), a screw feeder (not shown) transporting the metal powder (feedstock) in a fixed amount, a dispersion section (not shown) dispersing the metal powder (feedstock) transported by the screw feeder to convert it into the form of primary particles before the powder is finally sprayed, and a carrier gas supply source (not shown).

Together with a carrier gas to which a push-out pressure is applied from the carrier gas supply source, the metal powder (feedstock) is supplied into the thermal plasma flame 24 in the plasma torch 12 through the supply tube 14a.

The configuration of the material supply device 14 is not particularly limited as long as the device can prevent the metal powder (feedstock) from agglomerating, thus making it possible to spray the metal powder (feedstock) in the plasma torch 12 with the dispersed state maintained. Inert gas such as argon gas is used as the carrier gas, for example. The flow rate of the carrier gas can be controlled using, for instance, a flowmeter such as a float type flowmeter. The flow rate value of the carrier gas is a reading on the flowmeter.

The chamber 16 is provided below and adjacent to the plasma torch 12, and a gas supply device 28 is connected to the chamber 16. The primary fine particles 15 of the feedstock-based material (metal) are generated in the chamber 16. The chamber 16 serves as a cooling tank.

The gas supply device 28 is configured to supply cooling gas into the chamber 16. The gas supply device 28 includes a first gas supply source 28a, a second gas supply source 28b and piping 28c, and further includes a pressure application means (not shown) such as a compressor or a blower which applies push-out pressure to the cooling gas to be supplied into the chamber 16. The gas supply device 28 is also provided with a pressure control valve 28d which controls the amount of gas supplied from the first gas supply source 28a and a pressure control valve 28e which controls the amount of gas supplied from the second gas supply source 28b. For example, the first gas supply source 28a stores argon gas, while the second gas supply source 28b stores methane gas (CH₄ gas). In this case, the cooling gas is mixed gas of argon gas and methane gas.

The gas supply device 28 supplies the mixed gas of argon gas and methane gas as the cooling gas at, for example, 45 degrees in the direction of arrow Q toward a tail portion of the thermal plasma flame 24, i.e., the end of the thermal plasma flame 24 on the opposite side from the plasma gas supply port 12c, that is, a terminating portion of the thermal plasma flame 24, and also supplies the cooling gas from above to below along an inner wall 16a of the chamber 16, that is, in the direction of arrow R shown in FIG. 1.

The cooling gas supplied from the gas supply device 28 into the chamber 16 rapidly cools the feedstock (metal powder) having been converted to a gas phase state through the thermal plasma flame 24, thereby obtaining the primary fine particles 15 of the feedstock-based material (metal). Besides, the cooling gas has additional functions such as contribution to classification of the primary fine particles 15 in the cyclone 19. The cooling gas is, for instance, mixed gas of argon gas and methane gas.

When the primary fine particles 15 of the feedstock-based material (metal) having just been produced collide with each other to form agglomerates, this causes nonuniform particle size, resulting in lower quality. However, dilution of the primary fine particles 15 with the mixed gas which is supplied as the cooling gas in the direction of arrow Q toward the tail portion (terminating portion) of the thermal plasma flame prevents the fine particles from colliding with each other to agglomerate together.

In addition, the mixed gas supplied as the cooling gas in the direction of arrow R prevents the primary fine particles 15 from adhering to the inner wall 16a of the chamber 16 in the process of collecting the primary fine particles 15, whereby the yield of the produced primary fine particles 15 is improved.

Hydrogen gas may be added to the mixed gas of argon gas and methane gas used as the cooling gas. In this case, a third gas supply source (not shown) and a pressure control valve (not shown) that controls the amount of gas supply are further provided, and hydrogen gas is stored in the third gas supply source. For instance, hydrogen gas may be supplied by a predetermined amount in at least one of the directions of arrow Q and arrow R. Note that the cooling gas is not limited to argon gas, methane gas and hydrogen gas mentioned above.

The acid supply section 17 is configured to supply an organic acid to the primary fine particles 15 of the feedstock-based material (metal) (i.e., raw material fine particles) having been rapidly cooled by the cooling gas and thereby obtained. An organic acid supplied to a higher temperature region than the decomposition temperature of the organic acid is thermally decomposed and, on the primary fine particles 15 produced by rapidly cooling the thermal plasma having a temperature of about 10,000° C., the organic acid is deposited as an organic substance containing hydrocarbon (CnHm) and either a carboxyl group (—COOH) or a hydroxyl group (—OH) that provides hydrophilicity and acidity. Consequently, for instance, metal fine particles that are acidic properties can be obtained.

For example, the pH of the metal fine particles can be changed by changing the amount of the organic acid supplied to the primary fine particles 15 of the feedstock-based material (metal). For instance, even when the metal fine particles are certainly acidic, the degree of acidity, i.e., the acidity which is one surface property can be changed. The amount of the organic acid supplied can be changed using, for instance, the amount of an organic acid-containing aqueous solution supplied and the concentration of the organic acid.

The acid supply section 17 may have any configuration as long as it can provide an organic acid to the primary fine particles 15 of the feedstock-based material, e.g., the primary fine particles 15 of metal. For instance, an aqueous organic acid solution is used, and the acid supply section 17 sprays the aqueous organic acid solution into the chamber 16.

The acid supply section 17 includes a container (not shown) storing an aqueous organic acid solution (not shown) and a spray gas supply section (not shown) for converting the aqueous organic acid solution in the container into droplets. The spray gas supply section converts an aqueous solution into droplets using spray gas, and an aqueous organic acid solution AQ converted into droplets is supplied by a previously specified amount to the primary fine particles 15 of the feedstock-based material (metal) in the chamber 16. When the aqueous organic acid solution AQ is supplied (a step of supplying an organic acid), the atmosphere in the chamber 16 is an atmosphere in which the organic acid is thermally decomposed.

For the aqueous organic acid solution, pure water is used as the solvent, for instance. The organic acid is soluble in water, preferably has a low boiling point, and is preferably constituted of C, O and H only. Examples of the organic acid that may be used include L-ascorbic acid (C₆H₈O₆), formic acid (CH₂O₂), glutaric acid (C₅H₈O₄), succinic acid (C₄H₆O₄), oxalic acid (C₂H₂O₄), DL-tartaric acid (C₄H₆O₆), lactose monohydrate, maltose monohydrate, maleic acid (C₄H₄O₄), D-mannite (C₆H₁₄O₆), citric acid (C₆H₈O₇), malic acid (C₄H₆O₅) and malonic acid (C₃H₄O₄). The use of at least one of the foregoing organic acids is preferred.

For the spray gas used to convert the aqueous organic acid solution into droplets, argon gas is adopted for instance, but the spray gas is not limited to argon gas and may be inert gas such as nitrogen gas.

As shown in FIG. 1, the cyclone 19 is provided to the chamber 16 to classify the primary fine particles 15 of the feedstock-based material (metal) having been supplied with the organic acid, based on a desired particle size. The cyclone 19 includes an inlet tube 19a which supplies the primary fine particles 15 from the chamber 16, a cylindrical outer tube 19b connected to the inlet tube 19a and positioned at an upper portion of the cyclone 19, a truncated conical part 19c continuing downward from the bottom of the outer tube 19b and having a gradually decreasing diameter, a coarse particle collecting chamber 19d connected to the bottom of the truncated conical part 19c for collecting coarse particles having a particle size equal to or larger than the above-mentioned desired particle size, and an inner tube 19e connected to the collecting section 20 to be detailed later and projecting from the outer tube 19b.

A gas stream containing the primary fine particles 15 is blown in from the inlet tube 19a of the cyclone 19 along the inner peripheral wall of the outer tube 19b, and this gas stream flows in the direction from the inner peripheral wall of the outer tube 19b toward the truncated conical part 19c as indicated by arrow T in FIG. 1, thus forming a downward swirling stream.

When the downward swirling stream is inverted to an upward stream, coarse particles cannot follow the upward stream due to the balance between the centrifugal force and drag, fall down along the lateral surface of the truncated conical part 19c and are collected in the coarse particle collecting chamber 19d. Fine particles having been affected by the drag more than the centrifugal force are discharged to the outside of the system through the inner tube 19e along with the upward stream on the inner wall of the truncated conical part 19c.

The apparatus is configured such that a negative pressure (suction force) is exerted from the collecting section 20 to be detailed later through the inner tube 19e. The apparatus is also configured such that, under the negative pressure (suction force), the metal fine particles separated from the swirling gas stream are sucked as indicated by arrow U and sent to the collecting section 20 through the inner tube 19e.

On the extension of the inner tube 19e which is an outlet for the gas stream in the cyclone 19, the collecting section 20 for collecting the secondary fine particles (e.g., metal fine particles) 18 having a desired particle size on the order of nanometers is provided. The collecting section 20 includes a collecting chamber 20a, a filter 20b provided in the collecting chamber 20a, and a vacuum pump 30 connected through a pipe provided at a lower portion of the collecting chamber 20a. The fine particles delivered from the cyclone 19 are sucked by the vacuum pump 30 to be introduced into the collecting chamber 20a, remain on the surface of the filter 20b, and are collected.

It should be noted that the number of cyclones used in the production apparatus 10 is not limited to one and may be two or more.

Next, the fine particle production method using the production apparatus 10 above is described below taking metal fine particles as an example.

First, for example, metal powder having an average particle size of not more than 5 μm is charged into the material supply device 14 as the feedstock of the metal fine particles.

For example, argon gas and hydrogen gas are used as the plasma gas, and a high frequency voltage is applied to the coil 12b for high frequency oscillation to generate the thermal plasma flame 24 in the plasma torch 12.

Further, for instance, mixed gas of argon gas and methane gas is supplied as the cooling gas in the direction of arrow Q from the gas supply device 28 to the tail portion of the thermal plasma flame 24, i.e., the terminating portion of the thermal plasma flame 24. At that time, the mixed gas of argon gas and methane gas is also supplied as the cooling gas in the direction of arrow R.

Next, the metal powder is transported with gas, e.g., argon gas used as the carrier gas and supplied to the thermal plasma flame 24 in the plasma torch 12 through the supply tube 14a. The metal powder supplied is evaporated in the thermal plasma flame 24 to be converted into a gas phase state and is rapidly cooled with the cooling gas, thus producing the primary fine particles 15 of metal (metal fine particles). Further, the acid supply section 17 sprays a previously specified amount of aqueous organic acid solution in a droplet form to the primary fine particles 15 of metal.

Then, the primary fine particles 15 of metal thus obtained in the chamber 16 are blown in through the inlet tube 19a of the cyclone 19 together with a gas stream along the inner peripheral wall of the outer tube 19b, and accordingly, this gas stream flows along the inner peripheral wall of the outer tube 19b as indicated by arrow T in FIG. 1, thus forming a swirling stream which goes downward. When the downward swirling stream is inverted to an upward stream, coarse particles cannot follow the upward stream due to the balance between the centrifugal force and drag, fall down along the lateral surface of the truncated conical part 19c and are collected in the coarse particle collecting chamber 19d. Fine particles having been affected by the drag more than the centrifugal force are discharged along with the upward stream on the inner wall of the truncated conical part 19c from the inner wall to the outside of the system.

Due to the negative pressure (suction force) applied by the vacuum pump 30 through the collecting section 20, the discharged secondary fine particles (metal fine particles) 18 are sucked in the direction indicated by arrow U in FIG. 1 and sent to the collecting section 20 through the inner tube 19e to be collected on the filter 20b of the collecting section 20. The internal pressure of the cyclone 19 at this time is preferably equal to or lower than the atmospheric pressure. For the particle size of the secondary fine particles (metal fine particles) 18, an arbitrary particle size on the order of nanometers is specified according to the intended purpose.

Thus, the metal fine particles that are acidic can be easily and reliably obtained by merely subjecting the metal powder to plasma treatment and, for instance, spraying an aqueous organic acid solution thereto.

While the primary fine particles of metal are formed using a thermal plasma flame, the primary fine particles of metal may be formed by a gas-phase process. Thus, the method of producing the primary fine particles of metal is not limited to a thermal plasma process using a thermal plasma flame as long as it is a gas-phase process, and may alternatively be one using a flame process.

Furthermore, the metal fine particles produced by the method of producing metal fine particles according to this embodiment have a narrow particle size distribution, in other words, have a uniform particle size, and coarse particles of 1 μm or more are hardly included.

The flame process herein is a method of synthesizing fine particles by using a flame as the heat source and putting metal feedstock through the flame. In the flame process, the metal powder (feedstock) is supplied to a flame, and then cooling gas is supplied to the flame to decrease the flame temperature and thereby suppress the growth of metal particles, thus obtaining the primary fine particles 15 of metal. In addition, a previously specified amount of organic acid is supplied to the primary fine particles 15 to thereby produce the metal fine particles.

For the cooling gas and the organic acid, the same gases and acids as those mentioned for the thermal plasma flame described above can be used.

Aside from the metal fine particles described above, when such fine particles as oxide fine particles, nitride fine particles, carbide fine particles, oxynitride fine particles and resin fine particles mentioned above are produced, those fine particles such as oxide fine particles, nitride fine particles, carbide fine particles, oxynitride fine particles and resin fine particles can be produced in the same manner as the metal fine particles by using oxide powder, nitride powder, carbide powder, oxynitride powder and resin powder as the feedstock.

In production of fine particles other than the metal fine particles, gases and an organic acid appropriate for the composition are suitably used as the plasma gas, the cooling gas and the organic acid.

Next, the fine particles are described.

The fine particles of the invention are those called nanoparticles with a particle size of, for example, 1 to 100 nm. The particle size is the average particle size measured using the BET method. The fine particles of the invention are produced by, for instance, the production method described above and obtained in a particulate form. Thus, the fine particles of the invention are not present in a dispersed form in a solvent or the like but present alone. Therefore, there is no particular limitation on the combination of a solvent and the like, and the degree of freedom is high in selection of a solvent.

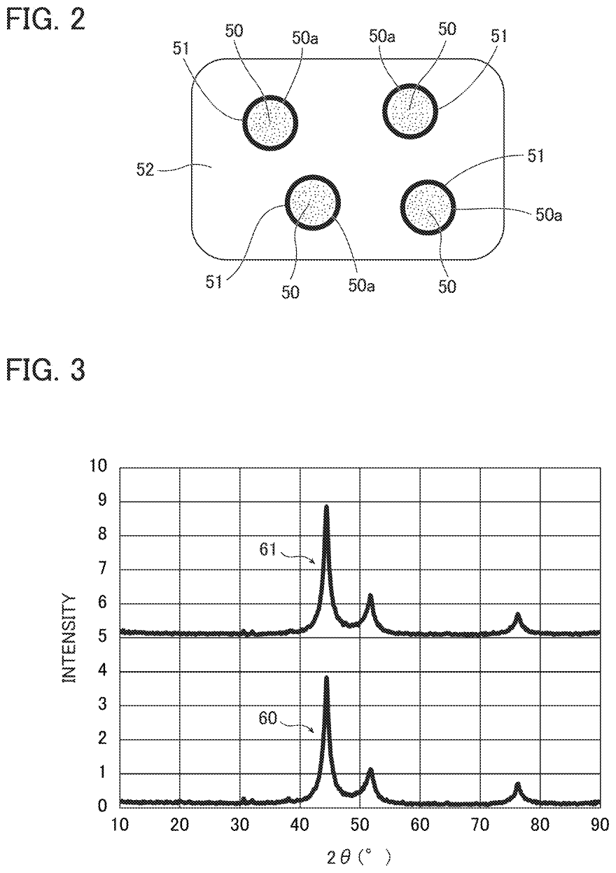

As shown in FIG. 2, each fine particle 50 has a surface coating 51 on its surface 50a. For metal fine particles as the fine particles 50 for instance, the surface condition of the particles including the surface coatings on their surfaces was examined, and the results suggesting that hydrocarbon (CnHm) was present on the surfaces and in addition to the hydrocarbon (CnHm), a hydroxyl group (—OH) or a carboxyl group (—COOH) that provides hydrophilicity and acidity was obviously present, were obtained.

The surface coating 51 is constituted of an organic substance that is generated by thermal decomposition of the organic acid and that contains hydrocarbon (CnHm) and either a carboxyl group (—COOH) or a hydroxyl group (—OH) which provides hydrophilicity and acidity. For example, the surface coating is constituted of an organic substance generated by thermal decomposition of citric acid.

While the surface coating 51 contains a hydroxyl group and a carboxyl group as described above, it suffices if the surface coating 51 contains, of a hydroxyl group and a carboxyl group, at least a carboxyl group.

When the surface condition of conventional metal fine particles was examined, the presence of hydrocarbon (CnHm) was confirmed, but such a result clearly suggesting the presence of a hydroxyl group and a carboxyl group was not obtained.

The surface condition of the fine particles 50 can be examined using, for instance, an FT-IR (Fourier transform infrared spectrometer).

When the pH of the metal fine particles that are one example of the fine particles of the invention and the pH of the conventional metal fine particles were obtained, the metal fine particles had a pH of 3.0 to 4.0, while the conventional metal fine particles had a pH of about 5 to about 7, as shown later. Thus, the pH of the fine particles can be controlled to the acidic side, and the acidity which is one surface property of the fine particles can be controlled. Therefore, it is possible to provide the fine particles with their surface properties such as the pH being controlled.

### <pH of Metal Fine Particles>

The pH of the metal fine particles can be measured as follows.

First, a specified amount of metal fine particles are charged in a container, and pure water (20 milliliters) is added dropwise to the fine particles and left to stand for 120 minutes. Then, the pH of a pure water part is measured. The pH is measured by a glass electrode method.

Note that the pH of fine particles other than the metal fine particles can be measured in the foregoing manner.

As described above, the metal fine particles of the invention have a more acidic property than the conventional metal fine particles. Accordingly, when the metal fine particles are dispersed in a solution 52 like the fine particles 50 shown in FIG. 2, only a small amount of basic dispersant (not shown) is required to have a necessary dispersed state.

Since a necessary dispersed state can be established with a small amount of basic dispersant, a coating film can be formed with a smaller amount of dispersant.

For the dispersant, for example, BYK-112 (BYK Japan KK) or the like may be used.

Next, specific examples of the fine particles are described taking metal fine particles as examples.

Sn fine particles (Sample 1) were produced using Sn (tin) powder as the raw material. For the Sn fine particles (Sample 1), an aqueous solution containing citric acid (citric acid concentration: 30 W/W %) was sprayed to primary fine particles of Sn with a spray gas. Argon gas was used as the spray gas.

Ni fine particles (Sample 3) were produced using Ni (nickel) powder as the raw material. For the Ni fine particles (Sample 3), an aqueous solution containing citric acid (citric acid concentration: 30 W/W) was sprayed to primary fine particles of Ni with a spray gas. Argon gas was used as the spray gas.

For comparison, Sn fine particles (Sample 2) and Ni fine particles (Sample 4) were produced using Sn (tin) powder and Ni (nickel) powder as the raw materials, respectively, by a conventional production method in which no organic acid was supplied.

The production conditions of the metal fine particles were as follows. Plasma gas: argon gas (200 liters/minute), hydrogen gas (5 liters/minute); carrier gas: argon gas (5 liters/minute); rapidly-cooling gas: argon gas (900 liters/minute), methane gas (10 liters/minute); internal pressure: 40 kPa.

The particle size of the fine particles thus obtained was measured by the BET method. As can be seen in Table 1 below, with the method of producing the metal fine particles according to the invention, the pH can be controlled to the acidic side.

TABLE 1

| | | Particle size | |
|---|---|---|---|
| | Type | (nm) | pH |
| Sample 1 | Sn | 231 | 3.7 |
| Sample 2 | Sn | 69 | 5.1 |
| Sample 3 | Ni | 21 | 3.0 |
| Sample 4 | Ni | 7 | 6.3 |

For the Ni fine particles of Samples 3 and 4, the crystal structures were analyzed by X-ray diffractometry. The results thereof are shown in FIG. 3. FIG. 3 is a graph showing analysis results of the crystal structures of the metal fine particles obtained by the production method of the invention and the metal fine particles obtained by the conventional production method, as analyzed by X-ray diffractometry. The unit of the intensity on the vertical axis is dimensionless.

In FIG. 3, numeral 60 represents a spectrum of the Ni fine particles (Sample 3) obtained by the fine particle production method of the invention, and numeral 61 represents a spectrum of the Ni fine particles (Sample 4) obtained by the conventional fine particle production method, i.e., the method in which no organic acid was supplied.

As shown in FIG. 3, the spectrum 60 of Sample 3 and the spectrum 61 of Sample 4 are the same, and Sample 3 and Sample 4 are different only in the pH. It is clear also from this fact that the pH of the metal fine particles can be controlled with the fine particle production method of the invention.

The present invention is basically configured as above. While the fine particle production method and the fine particles according to the invention are described above in detail, the invention is by no means limited to the foregoing embodiments and it should be understood that various improvements and modifications are possible without departing from the scope and spirit of the invention.

### REFERENCE SIGNS LIST

10 fine particle production apparatus
12 plasma torch
14 material supply device
15 primary fine particle
16 chamber
17 acid supply section
18 fine particle (secondary fine particle)
19 cyclone
20 collecting section
22 plasma gas supply source
24 thermal plasma flame
28 gas supply device
30 vacuum pump
50 fine particle
51 surface coating

1-13. (canceled)

14. A fine particle production method for producing fine particles using feedstock by means of a gas-phase process, the method comprising:
a step of supplying an organic acid to raw material fine particles.

15. The fine particle production method according to claim 14,
wherein the gas-phase process is a thermal plasma process or a flame process.

16. The fine particle production method according to claim 14,
wherein in the step of supplying an organic acid, an aqueous solution containing the organic acid is sprayed to an atmosphere in which the organic acid is thermally decomposed.

17. The fine particle production method according to claim 15,
wherein in the step of supplying an organic acid, an aqueous solution containing the organic acid is sprayed to an atmosphere in which the organic acid is thermally decomposed.

18. The fine particle production method according to claim 14,
wherein the organic acid consists only of C, O and H.

19. The fine particle production method according to claim 15,
wherein the organic acid consists only of C, O and H.

20. The fine particle production method according to claim 14,
wherein the organic acid is at least one of L-ascorbic acid, formic acid, glutaric acid, succinic acid, oxalic acid, DL-tartaric acid, lactose monohydrate, maltose monohydrate, maleic acid, D-mannite, citric acid, malic acid and malonic acid.

21. The fine particle production method according to claim 15,
wherein the organic acid is at least one of L-ascorbic acid, formic acid, glutaric acid, succinic acid, oxalic acid, DL-tartaric acid, lactose monohydrate, maltose monohydrate, maleic acid, D-mannite, citric acid, malic acid and malonic acid.

22. The fine particle production method according to claim 14,
wherein the feedstock is powder of a metal other than silver, and metal fine particles are produced by means of the gas-phase process.

23. The fine particle production method according to claim 15,
wherein the feedstock is powder of a metal other than silver, and metal fine particles are produced by means of the gas-phase process.

24. Fine particles each having a surface coating,
wherein the surface coating contains at least a carboxyl group.

25. The fine particles according to claim 24,
wherein the fine particles have a particle size of 1 to 100 nm.

26. Fine particles each having a surface coating,
wherein the surface coating is constituted of an organic substance generated by thermal decomposition of an organic acid.

27. The fine particles according to claim 26,
wherein the fine particles have a particle size of 1 to 100 nm.

28. The fine particles according to claim 26,
wherein the organic acid is at least one of L-ascorbic acid, formic acid, glutaric acid, succinic acid, oxalic acid, DL-tartaric acid, lactose monohydrate, maltose monohydrate, maleic acid, D-mannite, citric acid, malic acid and malonic acid.

29. The fine particles according to claim 26,
wherein the organic acid is citric acid.

30. The fine particles according to claim 24,
wherein the fine particles are fine particles of a metal other than silver.

31. The fine particles according to claim 26,
wherein the fine particles are fine particles of a metal other than silver.
